(19) United States
(12) Patent Application Publication
(10) Pub. No.: US 2003/0232982 A1
(43) Pub. Date: Dec. 18, 2003

(54) Method to prepare porphyrin nanoparticles and its application as oxidation catalyst

(76) Inventor: Xianchang Gong, Fresh Meadows, NY (US)

Correspondence Address: KAVA TECHNOLOGY, INC., 65-16 UTOPIA PARKWAY, FRESH MEDOWS, NY, 11365, US

(21) Appl. No.: 10/163,119

(22) Filed: Jun. 5, 2002

(51) Int. Cl. C07D 487/22

(52) U.S. Cl. 540/145

(57) ABSTRACT

The present invention relates to a method to prepare porphyrin nanoparticles and its application as oxidation catalyst. Mixing solvent techniques was used to prepare porphyrin nanoparticles. The sizes of the resulted porphyin nanoparticles are in the range of 1-1000 nm. The resulted porphyrin nanoparticles were characterized by DLS, AFM and UV-Vis. The nanoparticles are stable in air from weeks to months and has been successfully transferred to Al₂O₃ and silica gel surface. The present invention also related to a method to use nanoparticles of porphyrins as catalyst for epoxidation reactions of olefins and hydroxylation reactions of saturated hydrocarbons. By loading the nanoparticles onto the surface of Al₂O₃ and silica gel, excellent catalytic activities were obtained in oxidation reactions.

### FIELD OF THE INVENTION

The present invention relates generally to a synthesis of porphyrin nanoparticles and its application as catalyst for oxidation reactions. More specifically, the invention relates to a method using mixing solvent techniques to prepare porphyrin nanoparticles and to use the resulted nanoparticles as catalyst for epoxidation reactions of olefins and hydroxylation reactions of saturated hydrocarbons.

### REFERENCE CITED

Mlodnicka, T. “Metalloporphyrins as catalyst in autoxidation processes: A review”, J. Mol. Catal. 1986, 36, 205-242

Kadish, K. M.; Smith, K. M.; Guilard, R. Ed, The Porphyrin handbook, Academic press, 1999

Drain, C. M.; Nifiatis, F.; Vasenko, A.; Batteas, J. D., “Porphyrin tessellation by design: Metal mediated self-assembly of large arrays and tapes”, Angew. Chem., Int. Ed. Engl. 1998, 37, 2344-2347

Drain, C. M.; Gong, X. “Synthesis of meso substituted porphyrins in air without solvents or catalysts”, J. Chem. Soc., Chem. Commun. 1997, 2117-2118

Keuren, E. V.; Georgieva, E.; Adrian, J. “Kinetics of the Formation of Organic Molecular Nanocrystals”, Nano Letters, 2001, 1(3), 141-144

Periasamy, N., “J- and H-Aggregates of Porphyrin-Surfactant Complexes: Time-Resolved Fluorescence and Other Spectroscopic Studies”, J. Phys. Chem. B, 1998, 102, 1528-1538

Hunter, C. A.; Sanders, J. K. M. “The nature of .pi.-.pi. interactions”, J. Am. Chem. Soc. 1990, 112, 5525-34.

Kano, K.; Minamizono, H.; Kitae, T.; Negi, S. “Self-Aggregation of Cationic Porphyrins in Water. Can .pi.-.pi. Stacking Interaction Overcome Electrostatic Repulsive Force?”, J. Phys. Chem. A, 1997,101, 6118-6124.

Matile, S.; Berova, N.; Nakanishi, K., “Intramolecular porphyrin .pi.,.pi.-stacking: absolute configurational assignment of acyclic compounds with single chiral centers by exciton coupled circular dichroism”, Enantiomer, 1996, 1, 1-12.

Romeo, A., “Chiral H- and J-Type Aggregates of meso-Tetrakis(4-sulfonatophenyl) porphine on -Helical Polyglutamic Acid Induced by Cationic Porphyrins”, Inorganic Chemistry, 1998, 37, 3647-3648.

Xu, W.; Guo, H.; Akins, D. L. “Aggregation of Tetrakis(p-sulfonatophenyl)porphyrin within Modified Mesoporous MCM-41”, J. Phys. Chem. B, 2001, 105, 1543-1546.

### BACKGROUND

It is well documented that the chemistry of many nano-scaled particles is substantially different than the chemistry of bulk materials composed of the same atoms and/or molecules. Thus nano-scaled particles composed of porphyrins are expected to have significantly different chemical activities than the free porphyrins. Nanoparticles of catalytic porphyrins should have enhanced stability and catalytic rate. Nanometer-scale particles have been prepared by metals, metal oxides and other inorganic materials. Nanoparticles composed of organic molecules have been prepared. Organic molecules also have been used in the self-assembling process to prepare “soft” nanostructures such as spheres and tubes. One reason why porphyrins are suitable organic molecules to prepare nanometer-scale particles is that most porphyrins are solids at temperatures as high as 350° C. Porphyrin nanoparticles are promising components of advanced materials because of the rich porphyrin chemistry, stability, and proven catalytic activity. In analogy to the inorganic and other organic nanoparticles, it is expected that nanoparticles of porphyrins will have some special properties not obtainable by larger scaled materials.

The epoxidation of olefins is an important reaction, which is greatly used in organic synthesis. The current industrial method for the catalysis of olefin oxidation is by transition metal catalysts that are both expensive and harmful to the environment. Photochemical or chemical activation of O₂ will be the most environmentally friendly and economical oxidation method. The current photochemical epoxidation of olefins using porphyrin as a homogenous catalyst has poor efficiency due to the slow reaction speed (Mlodnicka, T. “Metalloporphyrins as catalyst in autoxidation processes: A review”, J. Mol. Catal. 1986, 36, 205-242). Similarly; saturated hydrocarbons are a promising raw material for the manufacture of chemicals. The discovery of new catalytic hydroxylation reactions, able to operate at mild temperature and heterogeneous is a challenging area in oxidation chemistry(Mlodnicka, T. “Metalloporphyrins as catalyst in autoxidation processes: A review”, J. Mol. Catal. 1986, 36, 205-242).

The present invention relates to a method for preparation of porphyrin nanoparticles and its application as oxidation catalyst. The resulted porphyrin nanoparticles show promising catalytic activity in epoxidation reactions of olefins and hydroxylation reactions of saturated hydrocarbons.

### SUMMARY OF THE INVENTION

The present invention relates to a method of preparation of porphyrin nanoparticles and its application as oxidation catalyst. More specifically, the invention relates to a method using mixing solvent techniques to prepare porphyrin nanoparticles. The sizes of the resulted porphyin nanoparticles are in the range of 1-1000 nm. The present invention also related to a method to use nanoparticles of porphyrins as catalyst for epoxidation reactions of olefins and hydroxylation reactions of saturated hydrocarbons.

### BRIEF DESCRIPTION OF THE DRAWINGS

FIG. 1. DSL characterization of Fe-tetrakis[di(ethylene glycol)monomethyl-2-pyridium]porphyrin pentachloride nanoparticles

FIG. 2. AFM images of Fe-tetrakis[di(ethylene glycol)monomethyl-2-pyridium]porphyrin pentachloride nanoparticles on glass in air

FIG. 3. Histograms of Fe-tetrakis[di(ethylene glycol)monomethyl-2-pyridium]porphyrin pentachloride nanoparticles on glass in air taken by topographic AFM measurements.

FIG. 4. UV-Vis. Spectra of 5,10,15,20-Tetrakis(4-carboxyl)porphyrin in DMASO (a) and its nanoparticles in water (b).

FIG. 5. Catalytic activity of porphyrin nanoparticles in the epoxidation reaction of cyclohexene. Molar ratios of porphyrin:PhIO:substrate is 1:1146:25189, nanoparticles loading is 0.1 wt % on the solid support. Yields and turnover numbers were determined by GC. (a) Porphyrin nanoparticles was absorbed onto alumina surface, Yield: 52%, TON:536; (b) Porphyrin nanoparticles was absorbed onto silica gel surface. Yield: 37.5%, TON:321; (c) Porphyrin nanoparticles in CH₃CN, yield:1 1.3%, TON:86. Retention time: cyclohexene: 2.5 min., cyclohexene oxide: 5.2 min., cyclohexen-3-ol: 7.4 min., iodobenzene: 13.8 min.

### DETAILED DESCRIPTION OF THE INVENTION

The present invention related to a method to prepare nanoparticles of porphyrins and their application as oxidation catalyst. Nanoparticles of porphyrins can be prepared by mixing solvent techniques in high yield and narrow distribution of particle size. The size of the nanoparticles generated in this invention is within the range of 1-1000 nm.

Porphyrins are a class of organic molecules with a core structure of a 20 member ring and different substitutes, Formula I or II.

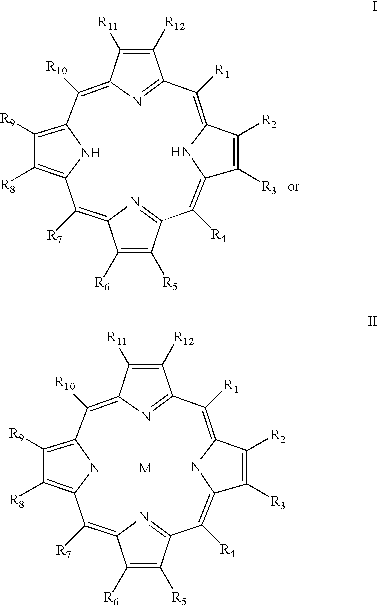

Wherein: R₁, R₂, R₃, R₄, R₅, R₆, R₇, R₈, R₉, R₁₀, R₁₁ and R₁₂ are substitutes on the porphyrin ring and M is a metal ion.

Porphyrins have remarkable photo-, catalytic-, electron, and bio-chemical properties (Kadish, K. M.; Smith, K. M.; Guilard, R. Ed, The Porphyrin handbook, Academic press, 1999). They have been extensively used in self-assembling processes to prepare nanometer-scale monolayers and thin films. Porphyrin arrays have been prepared both by organic synthesis and self-assembling techniques. Drain et al have reported the first nanomaterial composed solely of porphyrins (Drain, C. M.; Nifiatis, F.; Vasenko, A.; Batteas, J. D., “Porphyrin tessellation by design: Metal mediated self-assembly of large arrays and tapes”, Angew. Chem., Int. Ed. Engl. 1998, 37, 2344-2347).

Compared to the nanometer-scale particles compose of metals or metal oxides, porphyrin nanoparticles have both advantages and disadvantages. The advantages are listed below.

The high diversity of porphyrins and their isomers.

The methods of synthesis of porphyrins have been well developed (Drain, C. M.; Gong, X. “Synthesis of meso substituted porphyrins in air without solvents or catalysts”, J. Chem. Soc., Chem. Commun. 1997, 2117-2118).

The remarkably diverse photo-, catalytic-, electron, and bio-chemical properties of porphyrins has been well documented

Mixing solvents techniques has been used to prepare various nanoparticles (Keuren, E. V.; Georgieva, E.; Adrian, J. “Kinetics of the Formation of Organic Molecular A Nanocrystals”, Nano Letters, 2001, 1(3), 141-144). Nanoparticles composed of a few organic molecules have been successfully prepared by this method. In this invention, this method is used to prepare nanoparticles of porphyrins. Under controlled conditions (where mixing, temperature, and rate of solvent addition are critical factors) nano-scaled aggregates of rigid porphyrin molecules can be reproducibly made. The functionality of the nanoparticle will dictate the desired (or tolerable) functional groups on the porphyrins.

The method to prepare porphyrin nanoparticles in this invention is by dissolving one or a mixture of porphyrins in a host solvent, adding a stabilizer, followed by adding a guest solvent.

The host solvent used in the preparation method is one or a mixture of compounds that can dissolve porphyrins. For example, it can be one or a combination of these solvents: Dimethyl sulfoxide, N,N-Dimethylformamide, Tetrahydrofuran, Acetyl aldehyde, Acetonitrile, Methanol, Ethanol, Water, Chloroform, Dichloromethane, Carbon Tetrachloride, Toluene, Hexane, Propoinic acid, Acetic acid, Petroleum ether, Propylene oxide, Ethylene oxide, Ethyl ether, Xylene, Benzene, Acetone, Ethylacetate, cyclohexene, formaldehyde, cyclohexane, 1,4-dioxane, 1,2-Dichloroethane, Epichlorhydrin, Ethylene glycol, Methyl n-butyl ketone, Methyl chloride, Methyl ethyl ketone, Perchloroethylene, Styrene, 1,1,1-trichloroethane, Trichloroethylene, Pyridine.

The guest solvent in the preparation method is one or a mixture of compounds that is a liquid at room temperature. For example, it can be one or a combination of these solvents:

Dimethyl sulfoxide, N, N-Dimethylformamide, Tetrahydrofuran, Acetyl aldehyde, Acetonitrile, Methanol, Ethanol, Water, Chloroform, Dichloromethane, Carbon Tetrachloride, Toluene, Hexane, Propoinic acid, Acetic acid, Petroleum ether, Propylene oxide, Ethylene oxide, Ethyl ether, Xylene, Benzene, Acetone, Ethylacetate, cyclohexene, formaldehyde, cyclohexane, 1,4-dioxane, 1,2-Dichloroethane, Epichlorhydrin, Ethylene glycol, Methyl n-butyl ketone, Methyl chloride, Methyl ethyl ketone, Perchloroethylene, Styrene, 1,1,1-trichloroethane, Trichloroethylene, Pyridine.

The stabilizer in this invention is one or a mixture of compounds that can be dissolved in the host solvent. For example, it can be one or a mixture of polyethylene glycol derivative or a polyamine derivative. One choice of polyethylene glycol derivative is (CₘH₂ₘ₊₁)ⱼ(OCₙH₂ₙ₊₁)ₚOZ, where in group Z is —C_{q}H_{2q+1} or —C_{q}H_{2q}CH═CH₂, m, n, p, q=0-100. One choice of polyamine derivative is (CₘH₂ₘ₊₁)ⱼ(NHCₙH₂ₙ₊₁)ₚNH—Z, where in group Z is —CqH_{2q+1} or —C_{q}H_{2q}CH═CH₂, m, n, p, q=0-100. The amount of stabilizer adding to the host solvents is in the range of 0-100% volume ratio of the host solvent.

The resulted porphyrin nanoparticles are stable in air from weeks to months. The driving force for the formation of the porphyrin nanoparticles are likely due to π-stacking effects. Porphyrin pi-stacking has been known and studied for decades (Porphyrin pi-stacking: (a) Periasamy, N., “J- and H-Aggregates of Porphyrin-Surfactant Complexes: Time-Resolved Fluorescence and Other Spectroscopic Studies”, J. Phys. Chem. B, 1998, 102, 1528-1538 (b) Hunter, C. A.; Sanders, J. K. M. “The nature of.pi.-.pi. interactions”, J. Am. Chem. Soc. 1990, 112, 5525-34. (c) Kano, K.; Minamizono, H.; Kitae, T.; Negi, S. “Self-Aggregation of Cationic Porphyrins in Water. Can .pi.-.pi. Stacking Interaction Overcome Electrostatic Repulsive Force?”, J. Phys. Chem. A, 1997, 101, 6118-6124. (d) Matile, S.; Berova, N.; Nakanishi, K., “Intramolecular porphyrin .pi.,.pi.-stacking: absolute configurational assignment of acyclic compounds with single chiral centers by exciton coupled circular dichroism”, Enantiomer, 1996, 1, 1-12. (e) Romeo, A., “Chiral H- and J-Type Aggregates of meso-Tetrakis(4-sulfonatophenyl) porphine on -Helical Polyglutamic Acid Induced by Cationic Porphyrins”, Inorganic Chemistry, 1998, 37, 3647-3648.). These aggregates generally fall into two types, “J” and “H” where J refers to an edge-to-edge overlap arrangement of the porphyrins, and H refers to a face-to-face overlap. Each type, J and H, have distinctive spectral features that can be exploited or utilized. Heretofore, the controlled aggregation of porphyrins has been limited to encapsulation or molding strategies (Xu, W.; Guo, H.; Akins, D. L. “Aggregation of Tetrakis(p-sulfonatophenyl)porphyrin within Modified Mesoporous MCM-41”, J. Phys. Chem. B, 2001, 105, 1543-1546.), and to self-assembly methods (Drain, C. M.; Nifiatis, F.; Vasenko, A.; Batteas, J. D., “Porphyrin tessellation by design: Metal mediated self-assembly of large arrays and tapes”, Angew. Chem., Int. Ed. Engl. 1998, 37, 2344-2347). The latter uses metal ion coordination, hydrogen bonding, and electrostatic forces. Encapsulated aggregates may be of limited value as chemical catalysts. Discrete arrays of covalently bonded porphyrins and porphyrin polymers have also been known for over a decade. The extensive chemistry and very low yields of the discrete arrays, and the lack of control in the particle/polymer size limit the feasibility of using these in industrial-scale processes, though they are important for understanding the complex photo physics of multi chromophoric systems.

In a polar solvent such as water, π-stacking effect becomes stronger. The initial dispersion in the solvent system may contain hundreds of porphyrins π-stacked together. If the particle is more than one porphyrin wide in any dimension, the aggregation may be cooperative—resulting in more thermodynamically stable particles than expected from the ˜5 kcal/mol per porphyrin face pi-stacking energy. If the initial dispersion can be stabled by PEG surfactants, then the porphyrin nanoparticles can be characterized and their chemistry evaluated. A well known phenomena may help to understand this hypothesis. When porphyrins carrying positive charges interact with DNA, the cationic porphyrin molecules can aggregate along the side of DNA to form nano-scaled photonic materials. The PEG molecules serve to (1) inhibit nanoparticle agglomeration, (2) modify the surface chemistry, (3) determine the final particle size and distribution.

The present invention also related to a method of using nanoparticles of porphyrins as catalyst for oxidation reactions. Oxidation reactions include epoxidation reactions of olefins, hydroxylation reactions of saturated hydrocarbons, oxidation of aldehydes and many other important reactions.

Porphyrins have been effective in the photochemical, chemical, and electrochemical oxidation of hydrocarbons using dioxygen, iodosylbenzene, and applied potentials as oxidants (Mlodnicka, T. “Metalloporphyrins as catalyst in autoxidation processes: A review”, J. Mol. Catal. 1986, 36, 205-242). Porphyrin nanoparticles prepared in this invention effectively increases the catalyst turnover rate of oxidation reactions.

The oxidation catalyst in this invention can be made by absorbing nanoparticles of porphyrins to supports that have large surface area such as alumina or silica gel. The loading of the porphyrin nanoparticles on the support is between 0-100% wt ratios of the support used. The activity of the catalyst can be adjusted by the loading of the porphyrin nanoparticles on the support or by the size of the loaded porphyrin nanoparticles.

The operation temperature of the catalyst made by porphyrin nanoparticles in this invention is in the range of 0-700° C.

In this invention, UV-Visible spectrophotometer, Dynamic light scattering (DLS) and atomic force microscope (AFM) were used to characterize the nanoparticle size.

### EXAMPLES

The following chemicals were utilized in examples 1-12 that follow. 5,15-di(4-bromophenyl)-10,20-di(mesityl) porphyrin was also prepared according to published method(Littler, B. J.; Ciringh, Y.; Lindsey, J. S.; “Investigation of Conditions Giving Minimal Scrambling in the Synthesis of trans-Porphyrins from Dipyrromethanes and Aldehydes”, J. Org. Chem.; 1999; 64(8); 2864-2872). All other chemicals were purchased from Aldrich. DLS experiments were performed with a PD2000DLS (Precision Detector). AFM experiments were performed with Nanoscope IIIa Multi-probe microscope (Digital Instruments). All measurements were made in air at room temperature using standard Si₃N₄ tips (Veeco) with nominal tip radius of curvature 20-60 nm and spring constant of 0.12 N/m. UV-Vis absorption spectra were obtained on a Carey 1 spectrophotometer.

### Example 1

### Synthesis Nanoparticles of Fe-tetrakis[di(ethylene glycol)monomethyl-2-pyridium]porphyrin Pentachloride

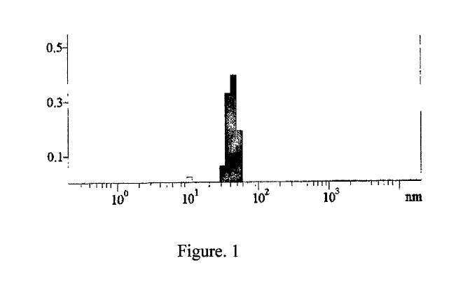

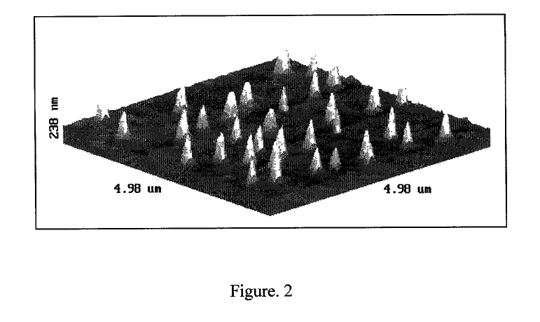

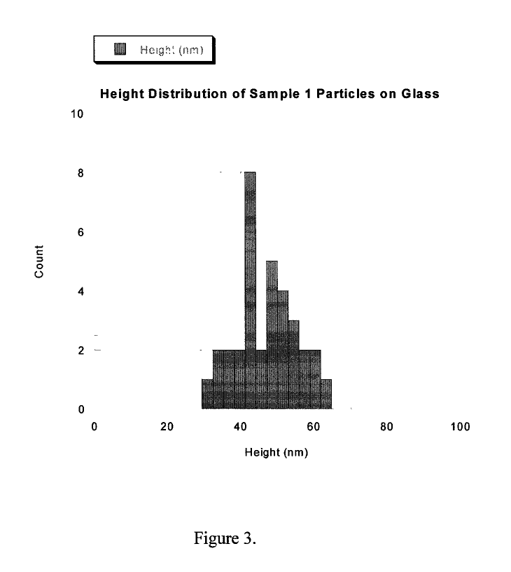

1 mg Fe-tetrakis[di(ethylene glycol)monomethyl-2-pyridium]porphyrin pentachloride was dissolved in 50 ul water followed by adding 5 ml acetonitrile. Yield: >95%. Average particle radius measure by DSL: 43.9 nm, FIG. 1. Sizes and distributions of the nanoparticles measured by AFM are in the range of 10 to 70 nm, FIG. 2. Histograms of nanoparticles on glass in air taken by topographic AFM measurements, FIG. 3.

### Example 2

### Synthesis Nanoparticles of 5,10,15,20-Tetrakis(4-carboxyl)porphyrin

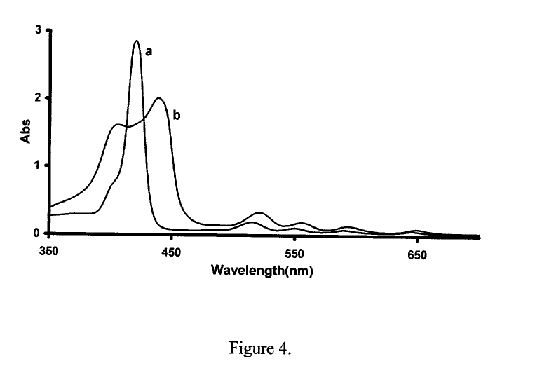

1.9 mg 5,10,15,20-Tetrakis(4-carboxyl)porphyrin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 46.6 nm. UV-Visible spectrum of porphyrin nanoparticles is clearly different compared to porphyrin in solution, FIG. 4.

### Example 3

### Synthesis Nanoparticles of 5,10,15,20-Tetrakis(4-methoxycarbonylphenyl) Porphyin

1.0 mg 5,10,15,20-Tetrakis(4-methoxycarbonylphenyl) porphyin was dissolved in 4 ml DMSO to make the stock solution. 0.4 ml stock solution was transferred to a test tube followed by adding 50ul Triethylene glycol monomethylether. After 1 minute, 5 ml water was added to this mixture and a glass rod was used to stir the mixture. Yield: >99%. Average particle radius measure by DSL: 58.2 nm.

### Example 4

### Synthesis Nanoparticles of 5,10,15,20-Tetrakisphenylporphyin

3.0 mg 5,10,15,20-Tetrakisphenylporphyin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 11.3 nm.

### Example 5

### Synthesis Nanoparticles of 5,10,15,20-Tetrakis(4-methoxyphenyl)porphyin

1.5 mg 5,10,15,20-Tetrakis(4-methoxyphenyl) porphyin porphyrin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 34.1 nm.

### Example 6

### Synthesis Nanoparticles of 5,10,15,20-Tetrakis(4-pyridyl)porphyin

0.7 mg 5,10,15,20-Tetrakis(4-pyridyl)porphyin was dissolved in 4 ml pyridine to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 70 nm.

### Example 7

### Synthesis Nanoparticles of 5,15-di(4-bromophenyl)-10,20-di(mesityl) Porphyrin

1.6 mg 5,15-di(4-bromophenyl)-10,20-di(mesityl) porphyrin was dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 24 nm.

### Example 8

### Synthesis Hybrid Nanoparticles of 5,10,15,20-Tetrakisphenylporphyin and 5,10,15,20-Tetrakis(4-methoxyphenyl) Porphyin

1.2 mg 5,10,15,20-Tetrakisphenylporphyin and 1.6 mg 5,10,15,20-Tetrakis(4-methoxyphenyl) porphyin were dissolved in 4 ml DMSO to make the stock solution. 0.4 ml stock solution was transferred to a test tube followed by adding 50 ul Triethylene glycol monomethylether. After 1 minute, 5 ml water was added to this mixture and a glass rod was used to stir the mixture. Yield: >99%. Average particle radius measure by DSL: 37.4 nm.

### Example 9

### Synthesis nanoparticles of 5,10,15,20-Tetrakisphenylporphyin Iron(III) Chloride

6 mg 5,10,15,20-Tetrakisphenylporphyin Iron(III) chloride was dissolved in 0.6 ml CH₂Cl₂ in a test tube followed by adding 50 ul Triethylene glycol monomethylether. After 1 minute, 5 ml water was added to this mixture and a glass rod was used to stir the mixture. Yield: >99%. Average particle radius measure by DSL: 12 nm.

### Example 10

### Synthesis Nanoparticles of 2,3,7,8,12,13,17,18-Octaethylporphyrin Magnesium(II)

0.6 mg 2,3,7,8,12,13,17,18-Octaethylporphyrin magnesium(II)was dissolved in 0.4 ml DMSO in a test tube followed by adding 50 ul NH₂(CH₂)₃NH(CH₂)₃NHC₁₁H₂₃. After 1 minute, 5 ml water was added to this mixture and a glass rod was used to stir the mixture. Yield: >99%. Average particle radius measure by DSL: 4.3 nm.

### Example 11

### Catalytic Activities of Nanoparticles of Fe-tetrakis[di(ethylene glycol)monomethyl-2-pyridium]porphyrin Pentachloride

Epoxidation reaction of cyclohexene was used to test the catalytic properties of porphyrin nanoparticles. The study was carried out in three different reaction conditions.

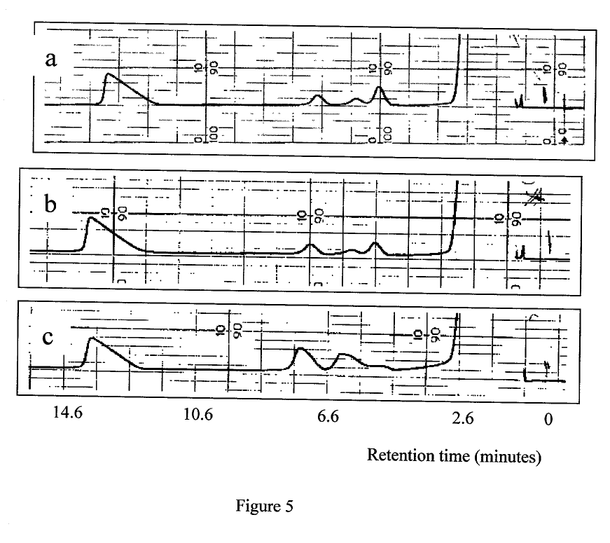

(a): 10 mmol cyclohexene, 0.455 mmol PhIO and 500 mg Al₂O₃/Fe-tetrakis[di(ethylene glycol)monomethyl-2-pyridium]porphyrin pentachloride nanoparticles catalyst (nanoparticles loading is 0.1% wt) was mixed in 5 ml CH₂Cl₂ and stirred at room temperature for 48 hours. Yield: 52%, Turnover numbers: 536. FIG. 5.

(b): 10 mmol cyclohexene, 0.455 mmol PhIO and 500 mg silica gel/Fe-tetrakis[di(ethylene glycol)monomethyl-2-pyridium]porphyrin pentachloride nanoparticles catalyst (nanoparticles loading is 0.1% wt) was mixed in 5 ml CH₂Cl₂ and stirred at room temperature for 48 hours. Yield: 37.5%, Turnover numbers: 321. FIG. 5

(c): 10 mmol substrate and 0.455 mmol PhIO were added to nanoparticles made by 3.96×10⁻⁴ mmol Fe-tetrakis[di(ethylene glycol)monomethyl-2-pyridium]porphyrin pentachloride in CH₃CN. The mixture was stirred at room temperature for 48 hours. Yield: 11.3%, Turnover numbers: 86. FIG. 5.

### Example 12

### Synthesis of Fe-tetrakis[di(ethylene glycol)monomethyl-2-pyridium]Porphyrin Pentachloride

Step1: 10.6 g 2-pyridinecarboxaldehyde and 6.7 g pyrrole were added to 1000 ml acetic acid in a 2 L flask. The mixture was refluxed for 2 hours. The solvent was removed under vacuum and the crude product was loaded onto a flash silica gel column. The column was developed by CHCl₃. 0.51 g pure 5,10,15,20-Tetrakis(2-pyridyl)porphyin was obtained.

Step2: 24 g diethylene glycol monomethylether and 30 g triethylamine were added to 1000 ml CH₂Cl₂. The mixture was cooled on ice. 38 g p-toluenesulfonyl chloride was added to the mixture. The mixture was stirred overnight at room temperature. The reaction mixture was cooled on ice for 3 hours to precipitate out the triethylamine hydrochloride salt. After filtration, the solvent was removed under vacuum to obtain the desired product. 30 g diethylene glycol monomethylether tosylate was made.

Step 3: 0.51 g pure 5,10,15,20-Tetrakis(2-pyridyl)porphyin, 30 g diethylene glycol monomethylether tosylate and 1 g FeCl₃ were added to 10 ml N,N-Dimethylformamide. The reaction was took place for 12 hours at 90° C. After the reaction was finished, water was added to extract the final product. 0.4 g Fe-tetrakis[di(ethylene glycol)monomethyl-2-pyridium]porphyrin pentachloride was made. UV-Visible spectrum in water: 407, 584. ESI-MS: 217[(M-5Cl⁻)⁵⁺/5].

1: A method to prepare nanoparticles of porphyrins: Dissolving one or a mixture of porphyrins in a host solvent, adding a stabilizer, followed by adding a guest solvent.

2: The porphyrins of claim 1 are a class of compounds has formula: 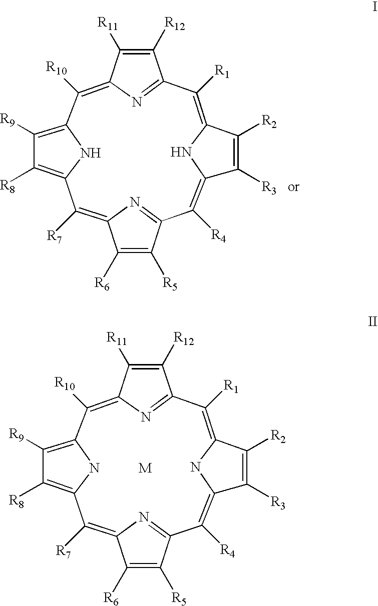
Wherein: R₁, R₂, R₃, R₄, R₅, R₆, R₇, R₈, R₉, R₁₀, R₁₁ and R₁₂ are substitutes on the porphyrin ring and M is a metal ion.

3: The sizes of nanoparticles of claim 1 are within the range of 1-1000 nm.

4: The host solvent of claim 1 is a solvent can dissolve porphyrins.

5: The host solvent of claim 4 is one or a mixture of these solvents: Dimethyl sulfoxide, N,N-Dimethylformamide, Tetrahydrofuran, Acetyl aldehyde, Acetonitrile, Methanol, Ethanol, Water, Chloroform, Dichloromethane, Carbon Tetrachloride, Toluene, Hexane, Propoinic acid, Acetic acid, Petroleum ether, Propylene oxide, Ethylene oxide, Ethyl ether, Xylene, Benzene, Acetone, Ethylacetate, cyclohexene, formaldehyde, cyclohexane, 1,4-dioxane, 1,2-Dichloroethane, Epichlorhydrin, Ethylene glycol, Methyl n-butyl ketone, Methyl chloride, Methyl ethyl ketone, Perchloroethylene, Styrene, 1,1,1-trichloroethane, Trichloroethylene, Pyridine.

6: The guest solvent of claim 1 is a solvent can form a solution with the host solvent.

7: The guest solvent of claim 6 is one or a mixture of these solvents: Dimethyl sulfoxide, N,N-Dimethylformamide, Tetrahydrofuran, Acetyl aldehyde, Acetonitrile, Methanol, Ethanol, Water, Chloroform, Dichloromethane, Carbon Tetrachloride, Toluene, Hexane, Propoinic acid, Acetic acid, Petroleum ether, Propylene oxide, Ethylene oxide, Ethyl ether, Xylene, Benzene, Acetone, Ethylacetate, cyclohexene, formaldehyde, cyclohexane, 1,4-dioxane, 1,2-Dichloroethane, Epichlorhydrin, Ethylene glycol, Methyl n-butyl ketone, Methyl chloride, Methyl ethyl ketone, Perchloroethylene, Styrene, 1,1,1-trichloroethane, Trichloroethylene, Pyridine.

8: The stabilizer of claim 1 is one or a mixture compounds can be dissolved in the host solvent.

9: The stabilizer of claim 8 is a polyethylene glycol derivative.

10: The polyethylene glycol derivative of claim 9 is (CₘH₂ₘ₊₁)ⱼ(OCₙH₂ₙ₊₁)ₚOZ, where in group Z is —C_{q}H_{2q+1} or —C_{q}H_{2q}CH═CH₂, m, n, p, j, q=0-100.

11: The stabilizer of claim 8 is a polyamine derivative.

12: The polyamine derivative of claim 11 is (CₘH₂ₘ₊₁)ⱼ(NHCₙH₂ₙ₊₁)ₚNH—Z, where in group Z is —C_{q}H_{2q+1} or —C_{q}H_{2q}CH═CH₂, m, n, p, j, q=0-100.

13: The stabilizer of claim 10 is triethylene glycol monomethylether.

14: M in the formula II of claim 2 is a metal ion selected from ions of a group of metals consisting of Fe, Mn, Co, Ni, Cu, Zn, Sn, Cr, V, Ru, Pt or Pd.

15: A method to use nanoparticles of porphyins as catalysts for oxidation reactions.

16: The nanoparticles of claim 15 are absorbed onto supports that have large surface area such as alumina or silica gel.

17: The porphyrins of claim 15 are a class of compounds has formula I or II: 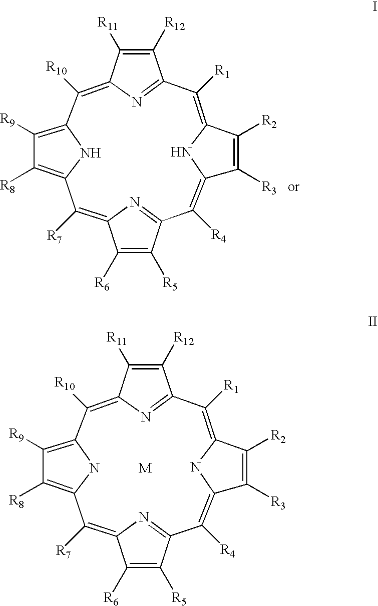
Wherein: R₁, R₂, R₃, R₄, R₅, R₆, R₇, R₈, R₉, R₁₀, R₁₁ and R₁₂ are substitutes on the porphyrin ring and M is a metal ion.

18: The nanoparticles of claim 15 is made by one porphyrin or a mixture of porphyrins.

19: The porphyrins of claim 17 are a class of compounds has formula III, IV or V: 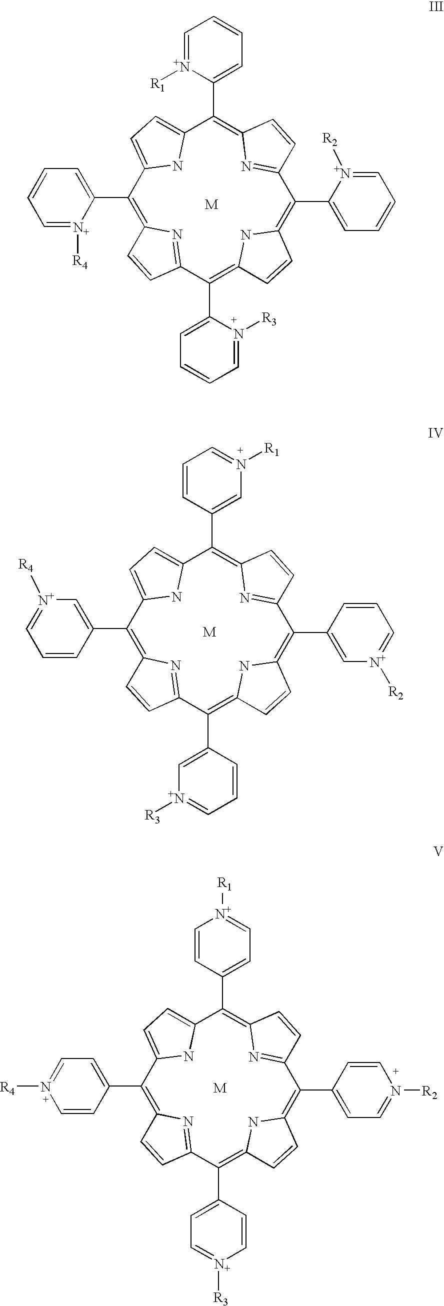
Wherein: R₁, R₂, R₃, R₄ are substitutes on the porphyrin ring and M is a metal ion.

20: M in the formula II of claim 17 is a metal ion selected from ions of a group of metals consisting of Fe, Mn, Co, Ni, Cu. Zn, Sn, Cr, V, Ru, Pt or Pd.

21: The sizes of nanoparticles of claim 15 are in the range of 0-100 nm.

22: The oxidation reactions of claim 15 are epoxidation reactions of olefins and hydroxylation reactions of saturated hydrocarbons.

23: The olefin of claim 22 is propylene
